(19) United States
(12) Patent Application Publication
(10) Pub. No.: US 2019/0151381 A1
(43) Pub. Date: May 23, 2019

(54) UPPER GASTROINTESTINAL FLORA-IMPROVING AGENT

(71) Applicants: MEIJI CO., LTD., Chuo-ku, Tokyo (JP); TOKAI UNIVERSITY EDUCATIONAL SYSTEM, Shibuya-ku, Tokyo (JP)

(72) Inventors: Yasuhiro Koga, Isehara-shi, Kanagawa (JP); Hirohiko Nakae, Isehara-shi, Kanagawa (JP); Toshihiro Ohtsu, Odawara-shi, Kanagawa (JP)

(21) Appl. No.: 16/308,526

(22) PCT Filed: Jun. 15, 2017
(86) PCT No.: PCT/JP2017/022065

§ 371 Date: Dec. 10, 2018

(30) Foreign Application Priority Data
Jun. 20, 2016 (JP) 2016-122155

(51) Int. Cl. A61K 35/747; A23L 33/135; A61P 1/14

(52) U.S. Cl. CPC A61K 35/747; A61P 1/14; A23L 33/135

(57) ABSTRACT

The problem is solved by an upper gastrointestinal flora-improving agent, the agent containing lactic acid bacteria as an effective ingredient, to be used in persons positive and negative for Helicobacter pylori. Lactic acid bacteria of the genus Lactobacillus is preferably used as the lactic acid bacteria, more preferably Lactobacillus gasseri OLL2716 (FERM BP-6999). Improvement of the upper gastrointestinal flora is, for example, a reduction in upper gastrointestinal Bifidobacterium and/or an increase in upper gastrointestinal Prevotella.

### TECHNICAL FIELD

The present invention relates to an upper gastrointestinal flora-improving agent for improving an upper gastrointestinal flora. Specifically, the present invention relates to an agent for decreasing the number of Bifidobacterium in upper gastrointestinal tract and/or an agent for increasing the number of Prevotella in upper gastrointestinal tract.

### BACKGROUND ART

Conventionally, in spite of progress in endoscopic diagnosis, there have been many cases where causes for complaints of upper gastrointestinal symptoms such as upper abdominal pain or discomfort, postprandial heavy stomach feeling, upper abdominal bloating, nausea, vomiting, epigastric pain, epigastric burning sensation or the like cannot be elucidated. Specifically, a state where in spite of presence of such complaints of digestive symptoms, no organic diseases can be found in general examination including endoscopic diagnosis and therefore no findings for elucidating the symptoms can be obtained is called FD (functional dyspepsia, unidentified complaints of upper abdomen, postprandial distress, epigastric pain, or functional gastroenteropathy),

Since these symptoms are not found as an organic disease, the symptoms are often overlooked or erroneously diagnosed. Therefore, the quality of life (QOL) of a person who has functional gastrointestinal disorders, which are accompanied by such discomfort as mentioned above but disease name of which is not identified in the diagnosis is lowered.

In attempting to improve these symptoms, it is known to administer serotonin or drugs releasing nitrogen monoxide. However, administration of such drugs causes side effects, and therefore it has been expected to prevent or improve functional gastrointestinal disorders by a method that does not cause side effects.

In attempting to prevent or improve functional gastrointestinal disorders by a method that does not cause side effects, various proposals have been made until now.

For example, it is described in Patent Document 1 and Patent Document 2 that functional gastrointestinal disorders can be improved by administering glutamic acid, 5′-nucleotide or the like (claim 1 of each document).

Patent Document 3 discloses an agent and food for prevention/amelioration of functional gastrointestinal disorders containing glutamic acid, arginine or the like as an effective ingredient. It is described therein that this agent can be produced easily at a low cost, and has high safety while being effective for upper gastrointestinal disorders such as functional dyspepsia (FD) (e.g., abdominal pains, heavy stomach feeling, and heart burn), and gastroesophageal reflux disease (GERD) (abstract of the document).

Further, as for a technology of preventing or improving functional gastrointestinal disorders by using lactic acid bacteria, Patent Document 4 discloses Bifidobacterium bifidum (hereinafter sometimes referred to as Bifidobacterium) that has an effect to remove Helicobacter pylori (hereinafter sometimes referred to as H. pylori) and exhibits high viability under aerobic conditions such as inside of fermented milk drinks. It is described therein that unidentified clinical syndrome of stomach can be improved by taking fermented milk drinks containing Bifidobacterium (paragraphs 0011 and 0096 of the document).

Patent Document 5 discloses that Lactobacillus gasseri MCC 1183 has an effect to remove H. pylori, and includes descriptions of anti-inflammatory agents and anti-ulcer drugs for use in preventing or treating gastritis utilizing this finding, and foods and drinks suitable for relieving heavy stomach feeling (paragraphs 0023 and 0052 of the document).

Patent Document 6 discloses a gastrointestinal function promotor containing lactic acid bacteria of the genus Lactobacillus such as Lactobacillus acidophilus (Lactobacillus gasseri) or the like, lactic acid bacteria of the genus Streptococcus such as Streptococcus faecalis, and aloe (claims 1 and 2, and paragraph 0012 of the document), and describes that promotion of gastrointestinal functions includes improvement of postprandial heavy stomach feeling and abdominal bloating (paragraph 0043 of the document).

As mentioned above, a technology of removing H. pylori and improving functional gastrointestinal disorders by using fermented milk drinks containing lactic acid bacteria such as Bifidobacterium has already been known in the art. In addition, removing H. pylori by using Lactobacillus gasseri MCC 1183 to relieve postprandial heavy stomach feeling has already been known, Further, improving postprandial heavy stomach feeling and abdominal bloating by using a gastrointestinal function promotor containing lactic acid bacteria of the genus Lactobacillus, lactic acid bacteria of the genus Streptococcus and aloe has already been proposed.

### RELATED ART DOCUMENTS

### Patent Documents

Patent Document 1: WO2006/030980

Patent Document 2: Japanese Patent No. 5067145

Patent Document 3: WO2009/113594

Patent Document 4: Japanese Patent No. 4881304

Patent Document 5: Japanese Patent No. 5300772

Patent Document 6: JP-A-2012-126700

Patent Document 7: Japanese Patent No. 4509250

Patent Document 8: WO2015/129281

### SUMMARY OF THE INVENTION

As described in Patent Document 7, the present applicant has a patent right for a Helicobacter pylori-removing medicament which is useful for removing H. pylori and/or preventing infection with H. pylori and which contains as an effective ingredient Lactobacillus gasseri l OLL2716 that has high capability of removing H. pylori. It is described therein that the medicament can be used as an anti-gastritis agent or an anti-ulcer agent (claims 1 and 3 of the document).

In addition, as described in Patent Document 8, the present applicant has discovered that Lactobacillus gasseri OLL2716 is effective for preventing or improving functional gastrointestinal disorders.

However, it has been unclear why Lactobacillus gasseri OLL2716 is effective as a prophylactic and/or therapeutic agent for functional gastrointestinal disorders. Specifically, the mechanism of prevention or improvement of functional gastrointestinal disorders using Lactobacillus gasseri OLL2716 has been unclear,

As a result of the present inventors' diligent research, it is discovered that Bifidobacterium are present in the stomachs in upper gastrointestinal tracts of patients having functional gastrointestinal disorders whereas Bifidobacterium are not present in the stomachs of healthy persons, and that Lactobacillus gasseri OLL2716 is effective for decreasing the number of Bifidobacterium in the upper gastrointestinal tracts.

In addition, it is discovered by the present inventors' diligent research that the number of Prevotella in the stomachs of patients having functional gastrointestinal disorders is less than that in the stomachs of healthy persons, and Lactobacillus gasseri OLL2716 is effective for increasing the number of Prevotella in the stomachs.

In this case, the present inventors have researched the effect of Lactobacillus gasseri OLL2716 on decrease in number of Bifidobacterium in the upper gastrointestinal tracts of patients and increase in number of Prevotella therein, and the research yields a remarkable result such that the effects are unrelated to eradication of H. Pylori caused by Lactobacillus gasseri OLL2716.

Further, it has also been discovered that the effects of Lactobacillus gasseri OLL2716, i.e., decrease in number of Bifidobacterium in the upper gastrointestinal tracts of patients and increase in number of Prevotella therein, can be produced even on persons negative for H. pylori.

Namely, it has been discovered that Lactobacillus gasseri OLL2716 can produce the effect to decrease the number of Bifidobacterium in the upper gastrointestinal tract and the effect to increase the number of Prevotella therein on persons positive for H. pylori as well as persons negative for H. pylori.

It is common technical knowledge that Bifidobacterium generally exist in bowels but do not exist in upper gastrointestinal tracts such as stomachs without mentioning that Bifidobacterium do not exist in stomachs of patients having functional gastrointestinal disorders unrelated to organic diseases. However, the research of the present inventors produces knowledge defying the common technical knowledge. Further, it is also common technical knowledge that the number of Prevotella in upper gastrointestinal tracts (such as stomachs) of persons does not decrease even when the persons have functional gastrointestinal disorders unrelated to organic diseases, but the research of the present inventors also produces knowledge defying the common technical knowledge,

The present invention has been made in view of the above-mentioned circumstances in the art, and the purpose of the invention is to provide an upper gastrointestinal flora-improving agent, which is for use in persons positive or negative for H. pylori. Specifically, the purpose of the invention is to provide an agent for decreasing the number of Bifidobacterium in upper gastrointestinal tract or an agent for increasing the number of Prevotella in upper gastrointestinal tract.

In order to accomplish the purpose, an upper gastrointestinal flora-improving agent is provided which is for use in persons positive H. pylori and persons negative for H. pylori and which includes lactic acid bacteria as an effective ingredient.

In the present invention, it is preferable to use lactic acid bacteria of the genus Lactobacillus as the lactic acid bacteria, and it is more preferable to use Lactobacillus gasseri OLL2716 (FERM BP-6999) as the lactic acid bacteria of the genus Lactobacillus.

Further, it is preferable that the agent be an upper gastrointestinal flora-improving agent for persons negative for H. pylori.

In the present invention, it is preferable that the number of lactic acid bacteria to be administered to a person per day is 2×10⁷ to 5×10¹⁰. When a cultured product of lactic acid bacteria is used and the number of lactic acid bacteria is 10⁷ or more per 1 g of the cultured product, it is preferable that the amount of the cultured product of lactic acid bacteria administered to a person per day is 5 g to 1000 g.

The upper gastrointestinal flora-improving agent of the present invention exhibits fast-acting properties. Specifically, the agent exhibits the upper gastrointestinal flora improving effect within 4 weeks after intake of the agent.

In this regard, specific examples of the upper gastrointestinal flora improvement include decrease in number of Bifidobacterium in upper gastrointestinal tract, and increase in number of Prevotella in upper gastrointestinal tract.

More specifically, specific examples of the upper gastrointestinal flora improvement include decrease in number of Bifidobacterium in stomach, and increase in number of Prevotella in stomach.

Further, it is preferable that these upper gastrointestinal flora-improving agents of the present invention are provided as foods, drinks, and functional foods such as health supplements, health-promoting foods and supplements.

According to the present invention, it is possible to provide an upper gastrointestinal flora-improving agent for both of persons positive for H. pylori and persons negative for H. pylori.

### DESCRIPTION OF THE EMBODIMENTS

Hereinafter, preferred embodiments of the present invention will be described in detail.

The upper gastrointestinal flora-improving agent according to the embodiment of the present invention is for use in persons positive for H. pylori and persons negative for H. pylori, and is characterized by including lactic acid bacteria as an effective ingredient.

According to the embodiment of the present invention, it is possible to provide an upper gastrointestinal flora-improving agent including, as an effective ingredient, lactic acid bacteria which are customarily eaten and hardly produce side effects, For example, according to the embodiment of the present invention, it is possible to provide an upper gastrointestinal flora-improving agent even to persons negative for H. pylori.

Lactic acid bacteria are generally used for fermented foods such as yogurt, cheese, butter and pickles, and some of lactic acid bacteria have familiar taste. Therefore, lactic acid bacteria can be easily taken.

According to the embodiment of the present invention, the genus, species and origin of lactic acid bacteria are not particularly limited as long as the lactic acid bacteria generate lactic acid by assimilating sugar. Among such lactic acid bacteria, lactic acid bacteria of the genus Lactobacillus is preferable, and particularly Lactobacillus gasseri is preferable. Among Lactobacillus gasseri, Lactobacillus gasseri OLL2716 (FERM BP-6999) can be preferably used.

In the embodiment of the present invention, the upper gastrointestinal flora means bacteria existing in upper gastrointestinal tract except for H. pylori. The inventors of the present invention have discovered that when a person having functional gastrointestinal disorders takes Lactobacillus gasseri OLL2716 (FERM BP-6999), the number of Bifidobacterium in the upper gastrointestinal tract of the person can be decreased and/or the number of Prevotella in the upper gastrointestinal tract can be increased to an extent such that the upper gastrointestinal flora of the person becomes similar to the upper gastrointestinal flora of healthy persons. It is common technical knowledge that Bifidobacterium generally exist in bowels but do not exist in upper gastrointestinal tracts such as stomachs without mentioning that Bifidobacterium do not exist in the stomachs of patients having functional gastrointestinal disorders unrelated to organic diseases. However, the research of the present inventors produces knowledge defying the common technical knowledge. Further, it is also common technical knowledge that the number of Prevotella in the upper gastrointestinal tracts of persons such as stomachs does not decrease even when the persons have functional gastrointestinal disorders unrelated to organic diseases, but the research of the present inventors also produces knowledge defying the common technical knowledge.

In the embodiment of the present invention, the functional gastrointestinal disorders mean the clinical condition of a person who does not suffer from organic diseases such as peptic ulcer or cancer but continuously has unidentified complaint in the upper gastrointestinal tract thereof, such as dyspepsia, heavy stomach feeling, abdominal bloating, nausea, vomiting, upper abdominal pain, poor appetite, and bowel movement disturbance. Namely, the functional gastrointestinal disorders mean that reproducible digestive symptoms that lower the QOL of a patient are observed even when the patient does not have an organic disease of gastrointestinal tract. Such functional gastrointestinal disorders are diseases that have been conventionally diagnosed as chronic gastritis or gastritis, and are characterized by showing symptoms such as abdominal pain, heavy stomach feeling and heartburn. In this regard, no organic diseases of gastrointestinal tract are found for patients having such functional gastrointestinal disorders, and therefore the disorders are considered to be caused by abnormal transmission in the nervous system due to stress or the like, presence of minor inflammations that cannot be detected by endoscopy, gastrointestinal hypomotility, or the like. However, the cause has not yet been clearly elucidated.

In this regard, the gastrointestinal tract means a series of hollow organs of from the oral cavity to the anus that are engaged in digestion. Specific examples of the gastrointestinal tract include pharynx, esophagus, stomach, small intestine (i.e., duodenum, jejunum and ileum), and large intestine. In addition, the upper gastrointestinal tract includes pharynx, esophagus, stomach and duodenum.

In the embodiment of the present invention, the dose of the upper gastrointestinal flora-improving agent is preferably as follows. Specifically, the number of lactic acid bacteria included, as an effective ingredient, in the upper gastrointestinal flora-improving agent administered to a person per day is preferably from 2×10⁷ to 5×10¹⁰, more preferably from 5×10⁷ to 5×10¹⁰, even more preferably from 1×10⁸ to 5×10¹⁰, still more preferably from 5×10⁸ to 5×10¹⁰, and even still more preferably from 5×10⁸ to 2×10¹⁰.

When the number of lactic acid bacteria included in the upper gastrointestinal flora-improving agent administered to a person per day is less than 2×10⁷, the effects to prevent and/or improve functional gastrointestinal disorders tend to be hardly produced. When the number of lactic acid bacteria is greater than 5×10¹⁰, no significant change can be observed in the effects.

When a cultured product of lactic acid bacteria is used for the upper gastrointestinal flora-improving agent and 10⁷ or more lactic acid bacteria are contained in 1 g of the cultured product, it is preferable that a person taking the agent can take the cultured product of lactic acid bacteria in an amount of from 5 g to 1000 g, more preferably from 10 g to 1000 g, even more preferably from 50 g to 500 g, still more preferably from 70 g to 300 g, even still more preferably from 70 g to 250 g, and most preferably from 80 g to 200 g per day. In this regard, in the embodiment of the present invention, the person can take the upper gastrointestinal flora-improving agent in a single dose or multiple doses per day (two or more doses per day).

The number of lactic acid bacteria contained in 1 g of a cultured product of lactic acid bacteria is not particularly limited as long as the number is 10⁷ or more, and therefore the number can be 10⁷, 10⁸, 10⁹ or the like. When the number of lactic acid bacteria contained in 1 g of the cultured product of lactic acid bacteria is increased, it becomes possible to decrease the amount of the cultured product included in the upper gastrointestinal flora-improving agent while including an effective dose of lactic acid bacteria in the agent. In this case, even when the amount of the cultured product of lactic acid bacteria that a person takes is smaller, the person can obtain the same effect as the effect to improve the upper gastrointestinal flora of persons mentioned above.

In the embodiment of the present invention, a cultured product of lactic acid bacteria can be obtained by culturing (proliferating) lactic acid bacteria with a known culture medium component. Further, by subjecting the thus obtained culture liquid to centrifugation or the like, a culture liquid that includes a greater number of lactic acid bacteria per unit weight of the culture liquid can be obtained. In the embodiment of the present invention, not only lactic acid bacteria in a state immediately after culturing (proliferating), but also lactic acid bacteria in a frozen state where lactic acid bacteria is mixed with a cryoprotective agent, and lactic acid bacteria in a frozen-dried state can be used. In the embodiment of the present invention, viable or killed lactic acid bacteria can be used, but viable lactic acid bacteria are preferable.

Commercially available products including lactic acid bacteria of the embodiment of the present invention can be used for convenience. For example, in a case of using Lactobacillus gasseri OLL2716 (FERM BP-6999), MEIJI PROBIO YOGURT LG21 manufactured and sold by Meiji Co., Ltd. can be used for convenience. It is possible to take the product itself, and it is also possible to use a processed product of the product. In the embodiment of the present invention, when lactic acid bacteria of the embodiment of the present invention are taken together with other ingestible components, no specific restrictions are imposed on the ingestible components, and, for example, milk components are preferably used as the ingestible components. In this regard, the milk components mean milk itself, and compositions which are prepared by processing milk and which include a milk constituent. Specific examples of such milk components include all the components which include a milk constituent, such as raw milk (e.g., cow milk), reconstituted milk (e.g., powder milk, cream and butter), cultured milk (e.g., yogurt and cheese), and milk preparation (e.g., milk whey, casein, lactose, whey mineral, and permeate), wherein the origin and form of the components are not particularly limited.

The upper gastrointestinal flora-improving agent according to the embodiment of the present invention has fast-acting properties. Particularly, the agent is characterized by producing the upper gastrointestinal flora improving effect within 4 weeks. Needless to say, it is not restricted to continuously take the agent for longer than 4 weeks. The upper gastrointestinal flora-improving agent according to the embodiment of the present invention is preferably taken continuously for 4 weeks or longer, more preferably for 8 weeks or longer, even more preferably for 12 weeks or longer, still more preferably for 16 weeks or longer, and even still more preferably for 20 weeks or longer.

The upper gastrointestinal flora-improving agent according to the embodiment of the present invention has no specific restriction on the ingestion method and the ingestion frequency. In one of the examples mentioned later, the upper gastrointestinal flora-improving agent is taken every day. In the embodiment mentioned above, a preferable number of lactic acid bacteria to be taken per day is described. However, it does not mean that the upper gastrointestinal flora improving effect according to the embodiment of the present invention cannot be produced unless the agent is certainly taken every day. Specifically, as long as the effect is produced, the ingestion frequency can be appropriately adjusted so that the agent is taken, for example, every other day, every three days, every four days, every five days, every seven days (once a week), every ten days, once a month, or the like.

The upper gastrointestinal flora-improving agent according to the embodiment of the present invention can be available as a unit dose package per one meal, The unit dose package of the agent can have a form such that an effective number of lactic acid bacteria are contained therein.

For example, it is preferable that lactic acid bacteria serving as an effective ingredient be contained in a unit dose package of the agent such that a person taking a dose of the agent can take lactic acid bacteria in number of from 2×10⁷ to 5×10¹⁰, more preferably from 5×10⁷ to 5×10¹⁰, even more preferably from 1×10⁸ to 5×10¹⁰, still more preferably from 5×10⁸ to 5×10¹⁰, and even still more preferably from 5×10⁸ to 2×10¹⁰.

Further, when the number of lactic acid bacteria contained in 1 g of a cultured product of lactic acid bacteria is 10⁷ or more, it is preferable that a person can take the cultured product of lactic acid bacteria serving as an effective ingredient preferably in an amount of from 5 g to 1000 g, more preferably from 10 g to 1000 g, even more preferably from 50 g to 500 g, still more preferably from 70 g to 300 g, even still more preferably from 70 g to 250 g, and most preferably from 80 g to 200 g per unit dose package.

When the upper gastrointestinal flora-improving agent according to the embodiment of the present invention is packed in the form of a unit dose package, any known packing materials can be used. Specific examples thereof include paper, plastics, glass, nylon, stainless steel, aluminum, iron, copper, silver, and bamboo, but are not limited thereto. In this regard, since lactic acid bacteria is facultative anaerobic bacteria, it is preferable to prevent lactic acid bacteria from contacting with air or oxygen, Therefore, it is preferable to provide a process to eliminate possibility of contact of lactic acid bacteria with oxygen in the process of manufacturing and/or packing the upper gastrointestinal flora-improving agent of the present invention. In addition, it is preferable to use a packing material for the package so that no oxygen is transmitted through the package during storage of the agent.

In the embodiment of the present invention, the method of taking the upper gastrointestinal flora-improving agent is not particularly limited, and any known methods such as oral intake methods, tube feeding methods, enteral tube feeding methods, injection methods through blood vessels, application methods, and methods using a suppository can be used. Among these methods, oral intake methods are preferably used.

In the embodiment of the present invention, when the upper gastrointestinal flora-improving agent is taken, the temperature of the agent is preferably from −30° C. to 50° C., more preferably from −20° C. to 45° C., even more preferably from 0° C. to 45° C., still more preferably from 0° C. to 30° C., even still more preferably from 0° C. to 20′C, and most preferably 0° C. to 10° C.

In the embodiment of the present invention, the upper gastrointestinal flora-improving agent can include components other than lactic acid bacteria, such as other edible components, various additives, foods, drinks, and raw materials of medicines.

Further, it is also preferable for the present invention to have a constitution such that the upper gastrointestinal flora-improving agent is provided as foods, drinks, functional foods such as health supplements, health-promoting foods, and supplements, or the like. In this regard, the functional foods mean foods having the third function (i.e., physical condition adjusting function) of the food functions, the health supplements mean health foods certified by Japan Health & Nutrition Food Association (JHFA), and health-promoting foods mean foods for specified health uses and foods with nutrient function claims that are approved by Japan Consumer Affair Agency. The foods and drinks mentioned above can include foods and drinks that do not fall into the category of functional foods. Further, in order that persons can continuously take the upper gastrointestinal flora-improving agent according to the embodiment of the present invention without being bored therewith, it is possible to process the upper gastrointestinal flora-improving agent into a form of drinks, yogurts, cheeses, and desserts. In this regard, the taste and physical properties of the upper gastrointestinal flora-improving agent can be changed so that the agent is suitable for the drinks and foods.

### EXAMPLES

Hereinafter, examinations performed to confirm the effects of the upper gastrointestinal flora-improving agent according to the embodiment of the present invention will be described. However, the present invention is not limited to the following constitutions.

### Example 1

A solid upper gastrointestinal flora-improving agent including Lactobacillus gasseri OLL2716 (FERM BP-6999) as an effective ingredient was prepared by the following method. Specifically, material milk, powdered skim milk and water were appropriately mixed such that the contents of milk fat and solid non-fat milk became 3.0% by weight and 9.2% by weight, respectively. After the mixture was homogenized by a normal method, the homogenized mixture was sterilized and cooled. Next, Lactobacillus bulgaricus, Streptococcus thermophilus and Lactobacillus gasseri OLL2716 (FERM BP-6999), which had been separated from MEIJI PROBIO YOGURT LG21 manufactured and sold by Meiji Co., Ltd., were inoculated thereinto, and the mixture was cultured by a normal method. The thus cultured product is hereinafter referred to as an upper gastrointestinal flora-improving agent of Example 1. In this regard, for the sake of convenience, the upper gastrointestinal flora-improving agent of Example 1 was prepared in such a manner that lactic acid bacteria included in the agent and including effective ingredients are to be directly taken by a person.

The number of Lactobacillus gasseri OLL2716 (FERM BP-6999) included in 1 g of the upper gastrointestinal flora-improving agent of Example 1 was about 10⁷.

(Test Method 1)

The upper gastrointestinal flora-improving agent of Example 1 (hereinafter sometimes referred to as a test sample) was subjected to an intervention trial.

Specifically, 24 patients who had no organic diseases and were negative for H. pylori and who had functional gastrointestinal disorders were trial subjects. In addition, a controlled study in which 21 healthy persons who had no organic diseases while being negative for H. pylori were targeted was also made.

In the intervention trial, the test sample was continuously given to each of the 24 patients for 12 weeks in a dose of 118 g per day.

In addition, the stomach fluids of the 24 patients, who had the above-mentioned functional gastrointestinal disorders, were directly obtained using a nasogastric tube before the intervention trial and after the patients continuously took the test sample for 12 weeks. In this regard, when the stomach fluids were obtained after the patients continuously had taken the test sample for 12 weeks, the stomach fluids were directly obtained from the stomachs one night after intake of the test sample. Similarly, the stomach fluids of the 21 healthy persons were also obtained directly from the stomachs of the healthy persons after a lapse of one night using a nasogastric tube.

(Identification of Flora in Stomach Fluid)

DNA of bacteria in the thus obtained stomach fluids was isolated by a normal method using UltraClean® Soil DNA Isolation Kit (from MO BIO Laboratories, Carlsbad, Calif.). The isolated DNA of bacteria was amplified by a PCR (polymerase chain reaction) method to determine the ratios of Bifidobacterium, Lactobacillus, Prevotella, Clostridium cluster IV, Clostridium subcluster XIVa, Clostridium cluster IX, Clostridium cluster XI, Clostridium cluster XVIII, and other bacteria.

(Bifidobacterium in Stomach Fluid)

When the ratio of Bifidobacterium in the stomach fluids was determined by the above-identified method, the ratio was 0.0% (n=21) for the 21 healthy persons. In contrast, the ratio was 2.5% (n=24) for the 24 patients having the above-mentioned functional gastrointestinal disorders before intake of the test sample, and was 0.4% (n=24) for the 24 patients after intake of the test sample.

When a significance test was performed to determine whether the ratio of Bifidobacterium (0.0% (n=21)) in the stomach fluids of the 21 healthy persons and the ratio of Bifidobacterium (2.5% (n=24)) in the stomach fluids of the 24 patients before intake of the test sample show a significant difference, the p-value was less than 0.001 and therefore it was judged that the ratios show a significant difference.

In addition, when a significance test was performed to determine whether the ratio of Bifidobacterium (0.0% (n=21)) in the stomach fluids of the 21 healthy persons and the ratio of Bifidobacterium (0.4% (n=24)) in the stomach fluids of the 24 patients after intake of the test sample show a significant difference, the p-value was less than 0.003 and therefore it was judged that the ratios show a significant difference.

Thus, it was found from the results that Bifidobacterium not present in the stomach fluids of the healthy persons were present in the stomach fluids of the patients having functional gastrointestinal disorders. In addition, it was also found that when the patients having functional gastrointestinal disorders continuously took the test sample, i.e., a yogurt containing Lactobacillus gasseri OLL2716 (FERM BP-6999), for 12 weeks, the number of Bifidobacterium in the stomach fluids of the patients could be decreased to such an extent that the conditions of the stomach fluids of the patients are close to those of the stomach fluids of the healthy persons.

(Prevotella in Stomach Fluid)

When the ratio of Prevotella in the stomach fluids was determined by the above-identified method, the ratio was 37.4% (n=21) for the 21 healthy persons. In contrast, the ratio was 28.3% (n=24) for the 24 patients having the above-mentioned functional gastrointestinal disorders before intake of the test sample, and was 39.8% (n=24) for the 24 patients after intake of the test sample.

When a significance test was performed to determine whether the ratio of Prevotella (37.4% (n=21)) in the stomach fluids of the 21 healthy persons and the ratio of Prevotella (28.3% (n=24)) in the stomach fluids of the 24 patients before intake of the test sample show a significant difference, the p-value was 0.004 and therefore it was judged that the ratios show a significant difference.

When a significance test was performed to determine whether the ratio of Prevotella (37.4% (n=21)) in the stomach fluids of the 21 healthy persons and the ratio of Prevotella (39.8% (n=24)) in the stomach fluids of the 24 patients after intake of the test sample show a significant difference, the p-value was 0.001 and therefore it was judged that the ratios show a significant difference.

Thus, it was found from the results that the ratio of Prevotella in the stomach fluids of the patients having functional gastrointestinal disorders is lower than the ratio of Prevotella in the stomach fluids of the healthy persons. In addition, it was also found that when the patients having functional gastrointestinal disorders continuously take the test sample, i.e., a yogurt containing Lactobacillus gasseri OLL2716 (FERM BP-6999), for 12 weeks, the number of Prevotella in the stomach fluids of the patients can be increased to such an extent that the conditions of the stomach fluids of the patients are close to those of the stomach fluids of the healthy persons.

Although only some exemplary embodiments and/or examples of this invention have been described above in detail, those skilled in the art will readily appreciate that many modifications are possible in the exemplary embodiments and/or examples without materially departing from the novel teachings and advantages of this invention. Accordingly, all such modification are intended to be included within the scope of this invention.

The documents described in this specification and the specification of a Japanese application on the basis of which the present application claims Paris Convention priority is incorporated herein by reference in its entirety.

1. An upper gastrointestinal flora-improving agent comprising lactic acid bacteria as an effective ingredient, wherein the upper gastrointestinal flora-improving agent is for use in persons positive for Helicobacter pylori and persons negative for Helicobacter pylori.

2. The upper gastrointestinal flora-improving agent according to claim 1, wherein the lactic acid bacteria is lactic acid bacteria of the genus Lactobacillus.

3. The upper gastrointestinal flora-improving agent according to claim 1, wherein the lactic acid bacteria is Lactobacillus gasseni OLL2716 (FERM BP-6999).

4. The upper gastrointestinal flora-improving agent according to claim 1, wherein the upper gastrointestinal flora-improving agent is for use in persons negative for Helicobacter pylori.

5. The upper gastrointestinal flora-improving agent according to claim 1, wherein a number of lactic acid bacteria to be administered to a person is from 2×10⁷ to 5×10¹⁰ per day.

6. The upper gastrointestinal flora-improving agent according to claim 1, wherein the lactic acid bacteria is a cultured product of lactic acid bacteria, and wherein when 10⁷ or more lactic acid bacteria are contained in 1 g of the cultured product of lactic acid bacteria, an amount of the cultured product of lactic acid bacteria to be administered to a person is from 5 g to 1000 g per day.

7. The upper gastrointestinal flora-improving agent according to claim 1, wherein the upper gastrointestinal flora-improving agent produces an upper gastrointestinal flora improving effect within 4 weeks after intake of the upper gastrointestinal flora-improving agent.

8. The upper gastrointestinal flora-improving agent according to claim 1, wherein the upper gastrointestinal flora improving effect is at least one of decrease in number of Bifidobacterium in upper gastrointestinal tract, and increase in number of Prevotella in upper gastrointestinal tract.

9. The upper gastrointestinal flora-improving agent according claim 1, Where in the upper gastrointestinal flora improving effect is at least one of decrease in number of Bifidobacterium in stomach, and increase in number of Prevotella in stomach.
